(19) United States
(12) Patent Application Publication
(10) Pub. No.: US 2014/0340217 A1
(43) Pub. Date: Nov. 20, 2014

(54) APPARATUS AND METHODS FOR SENSING A PARAMETER WITH A RESTRAINT DEVICE

(71) Applicant: Collar ID, LLC, Punta Gorda, FL (US)

(72) Inventors: Stephen P. Moenning, Punta Gorda, FL (US); Anne E. Moenning, Punta Gorda, FL (US); Emily J. Moenning, Punta Gorda, FL (US); John Acker, Fort Myers, FL (US)

(73) Assignee: Collar ID, LLC, Punta Gorda, FL (US)

(21) Appl. No.: 14/274,989

(22) Filed: May 12, 2014

Related U.S. Application Data
(60) Provisional application No. 61/823,622, filed on May 15, 2013.

(51) Int. Cl. A61B 5/0205; G08B 21/18; A61F 5/37; A61B 5/00; A61B 5/145

(52) U.S. Cl. CPC A61B 5/02055; A61B 5/441; A61B 5/14542; A61F 5/37; G08B 21/18
USPC 340/539.11; 600/300; 600/301; 600/549; 600/306; 600/508; 600/364

(57) ABSTRACT

A restraint device includes at least one sensor configured to sense at least one parameter associated with the wearer of the restraint device. The sensed parameter may be a physiological parameter of the wearer, or the sensed parameter may be a biological substance, a chemical substance, or a radioactive substance, for example. The restraint device may further include a device communicating with the sensor and configured to store and/or transmit information related to the sensed parameter via Radio Frequency Identification (RFID) communication.

### CROSS-REFERENCE TO RELATED APPLICATIONS

This application claims the priority of U.S. Provisional Application Serial Nos. 61/823,622, filed May 15, 2013 (pending). This application is also generally related to U.S. patent application Ser. No. 13/540,031, filed Jul. 2, 2012 (pending). The disclosures of each of these two prior applications are incorporated by reference herein.

### TECHNICAL FIELD

The present invention relates generally to devices and methods for acquiring information from a sensor associated with a restraint device.

### BACKGROUND

Various types of physical or electronic restraint devices are known for restraining the free movement of the wearer. Restraint devices may be used, for example, to restrain the movement of a person who has been involved in an accident, or who has otherwise been injured, to isolate the injury and prevent further harm to the individual. Examples of such restraint devices include cervical collars, knee braces, spinal braces, seat belts, and other similar devices.

In other situations, restraint devices may be used for law enforcement or other purposes, such as to restrict the free movement of persons who have been apprehended in connection with the commission of a crime, for example. Such restraint devices include, but are not limited to, handcuffs, leg shackles, waist chains, straightjackets, or electronic restraint devices.

Restraint devices may also be used for animal husbandry purposes, to restrict the free movement of the wearer, which may be a pet or other animal. For example, pets with electronic restraints on their collars are restrained within the confines of the owner's property. Animals that require preparations for breeding, for example, may also be restrained. Restraint devices may also be for security purposes, to restrict the free movement of persons who are attempting to pass through security check points such as airport security, for example. Such restraint devices include but are not limited to handcuffs in addition to whole body scanning. Restraint devices may also be used for shipping purposes, to restrict the free movement of cargo containers when shipped, for example.

In certain instances, it may be desirable to obtain information associated with the wearer, such as the individual, animal, or object that has been restrained with a restraint device. For example, when restraint devices are used for medical purposes, it may be desirable to sense at least one physiological parameter associated with the wearer of the device, such as skin temperature, skin pH, heart rate, the level of blood-oxygen saturation, a virulent bacterial status, or any other physiological parameter, as may be desired.

In other instances, it may be desirable to sense the presence of, or the exposure of the wearer to one or more substances, such as biological substances, chemical substances, or radioactive substances. For example, it may be desirable to determine whether the wearer of a restraint device has been exposed to, or has come in contact with, explosive materials, chemical weapons, radioactive materials, dangerous biological substances, illegal drugs, or other substances.

In other instances, it may be desirable to sense the presence of, or the exposure of restrained cargo containers when shipped to one or more substances, such as biological substances, chemical substances, or radioactive substances. For example, it may be desirable to determine whether a cargo container restrained by the restraint device when shipped includes explosive materials, chemical weapons, radioactive materials, dangerous biological substances, illegal drugs, or other substances.

Conventionally, when it has been desired to detect a physiological parameter associated with the wearer, or to determine if the wearer has been exposed to various biological, chemical, or radioactive substances such as those described above, additional steps must be taken to detect the desired physiological parameter or substance. In many instances, a sample must be obtained from the wearer and delivered to an appropriate laboratory so that tests may be conducted on the obtained sample. As a result, significant delays in obtaining information related to the restrained individual, and communicating that information to appropriate persons occur.

Conventionally, when it is desired to determine if the cargo container restrained by the restraint device includes various biological, chemical, or radioactive substances such as those described above, additional steps must be taken to detect the substance. In many instances, assessment of each cargo container of the presence of substances is obtained and recorded manually which is time consuming and cost prohibitive in the shipping industry.

A need therefore exists for a restraint device that overcomes these and other deficiencies of the prior art.

### SUMMARY

The present invention overcomes the foregoing and other shortcomings and drawbacks of restraint devices heretofore known for use in various medical, law enforcement, or other applications. While the invention will be described in connection with certain embodiments, it will be understood that the invention is not limited to these embodiments. On the contrary, the invention includes all alternatives, modifications and equivalents as may be included within the spirit and scope of the present invention.

According to one aspect of the present invention, a restraint device includes at least a first restraint portion defining a space for receiving at least a first body portion of a wearer to restrain movement of the wearer. The restraint device may restrain movement of the at least one body portion relative to another body portion of the wearer, or the restraint device may restrain movement of the at least one body portion relative to a permitted space. The restraint device further includes at least one sensor on the first restraint portion. The sensor is configured to sense at least one parameter associated with the wearer of the restraint device. The restraint device further includes a device for communicating with the at least one sensor and adapted to transmit to a remote location information related to the at least one sensed parameter. The device is also configured transmit information related to the at least one sensed parameter to a proximate communications device that is within proximity of the device to receive the transmitted information related to the at least one sensed parameter.

In one aspect, the sensor may be configured to sense at least one physiological parameter of the wearer, such as skin temperature, skin pH, heart rate, the level of blood oxygen saturation, a virulent bacterial status, or any other physiological parameter, as may be desired. Alternatively, or additionally, the sensor may be configured to sense the presence of, or the exposure of the wearer to, at least one of a biological substance, a chemical substance, or a radioactive substance. The restraint device may further include a device communicating with the sensor and configured to store information related to the sensed parameter. The device may additionally or alternatively be configured to transmit to a remote location information related to the sensed parameter. The device may additionally or alternatively be configured to transmit to proximate communications device that is within proximity of the device to receive the transmitted information related to the at least one sensed parameter.

These and other features, objects and advantages of the invention will become more readily apparent to those of ordinary skill in the art upon review of the following detailed description, taken in conjunction with the accompanying drawings.

### BRIEF DESCRIPTION OF THE FIGURES

Embodiments of the invention are described with reference to the accompanying drawings. In the drawings, like reference numbers indicate identical or functionally similar elements.

FIG. 1 depicts an exemplary restraint device in accordance with the principles of the present disclosure, including a sensor for detecting a parameter associated with the wearer of the device.

FIG. 2 depicts another exemplary restraint device in accordance with the principles of the present disclosure, in the form of handcuffs.

FIG. 3 depicts a third embodiment of an exemplary restraint device in accordance with the principles of the present disclosure, in the form of an ankle bracelet.

FIG. 4 depicts a fourth exemplary embodiment of a restraint device in accordance with the principles of the present disclosure, in the form of a cervical collar.

FIG. 5 depicts a block diagram of an exemplary embodiment of a communication configuration in accordance with the principles of the present disclosure, in the form of a restraint RFID device and a proximate communications device.

FIG. 6 depicts a block diagram of an exemplary embodiment of a restraint RFID device in accordance with the principles of the present disclosure.

FIG. 7 depicts a block diagram of an exemplary embodiment of a proximate communications device in accordance with the principles of the present disclosure.

FIG. 8 depicts a block diagram of an exemplary embodiment of transmitted sensor parameter configuration in accordance with the principles of the present disclosure.

The invention will now be described with reference to the accompanying drawings. In the drawings, like reference numbers indicate identical, functionally similar, and/or structurally similar elements.

### DETAILED DESCRIPTION

The following detailed description refers to accompanying drawings to illustrate exemplary embodiments consistent with the invention. References in the detailed description to “one exemplary embodiment,” “an exemplary embodiment,” “an example exemplary embodiment,” etc. indicate that the exemplary embodiment described may include a particular feature, structure, or characteristic, but every exemplary embodiment may not necessarily include the particular feature, structure, or characteristic. Moreover, such phrases are not necessarily referring to the same exemplary embodiment. Further, when a particular feature, structure, or characteristic is described in connection with an exemplary embodiment, it is within the knowledge of those skilled in the relevant art(s) to affect such feature, structure, or characteristic in connection with other exemplary embodiments whether or not explicitly described.

The exemplary embodiments described herein are provided for illustrative purposes, and are not limiting. Other exemplary embodiments are possible, and modifications may be made to the exemplary embodiments within the spirit and scope of the invention. Therefore, the detailed description is not meant to limit the invention. Rather, the scope of the invention is defined only in accordance with the following claims and their equivalents.

Embodiments of the invention may be implemented in hardware, firmware, software, or any combination thereof. Embodiments of the invention may also be implemented as instructions stored on a machine-readable medium, which may be read and executed by one or more processors. A machine-readable medium may include any mechanism for storing or transmitting information in a form readable by a machine (e.g., a computing device). For example, a machine-readable medium may include read only memory (ROM); random access memory (RAM); magnetic disk storage media; optical storage media; flash memory devices; electrical, optical, acoustical or other forms of propagated signals (e.g., carrier waves, infrared signals, digital signals, etc.), and others. Further firmware, software, routines, instructions may be described herein as performing certain actions. However, it should be appreciated that such descriptions are merely for convenience and that such actions in fact result from computing devices, processors, controllers, or other devices executing the firmware, software, routines, instructions, etc.

The following detailed description of the exemplary embodiments will so fully reveal the general nature of the invention that others can, by applying knowledge of those skilled in the relevant art(s), readily modify and/or adapt for various applications such as exemplary embodiments, without undue experimentation, without departing from the spirit and scope of the invention. Therefore, such adaptations and modifications are intended to be within the meaning and plurality of equivalents of the exemplary embodiments based upon the teaching and guidance presented herein. It is to be understood that the phraseology or terminology herein is for the purpose of description and not of limitation, such that the terminology or phraseology of the present specification is to be interpreted by those skilled in relevant art(s) in light of the teachings therein.

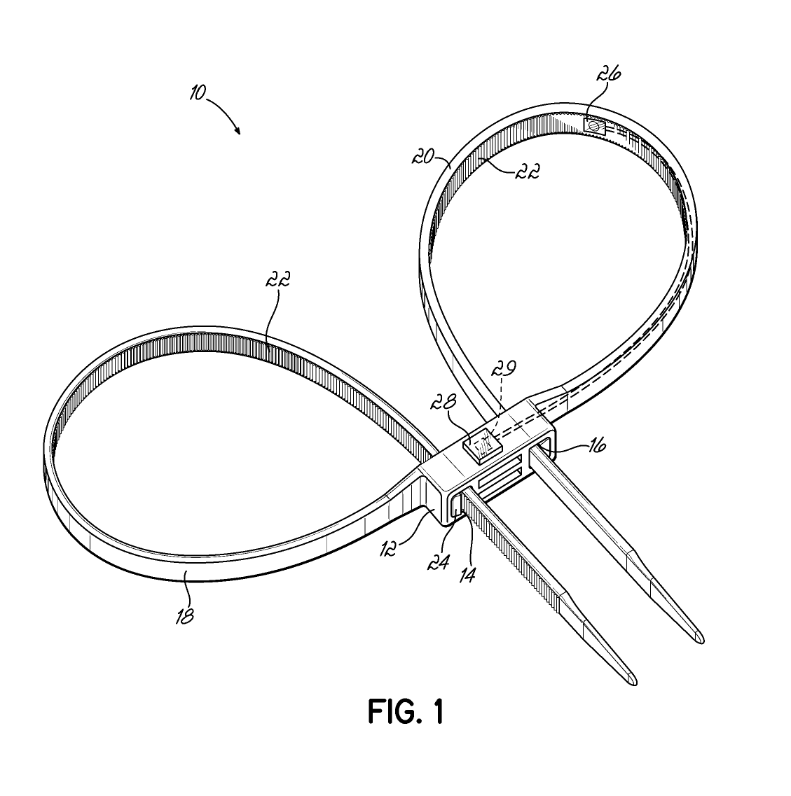

FIG. 1 depicts an exemplary restraint device 10 in the form of a double cuff restraint that may be applied to the wrist or ankles of a person to thereby restrain movement of the arms or legs of the person relative to one another. Such restraint devices 10 have been referred to as cable-tie or zip-tie type cuffs having a main body portion 12 with a pair of apertures 14, 16 through which respective first and second elongate straps 18, 20 may be directed to form loops or cuffs intended to surround the wrists or ankles of a person to be restrained. Each of the first and second elongate straps 18, 20 includes an integrally-formed gear rack having teeth 22 that can be engaged with a ratchet mechanism 24 disposed within the respective apertures 14, 16 to allow movement of the straps 18, 20 through the respective apertures 14, 16 in only one direction. Once the teeth 22 of the gear rack are engaged by the ratchet mechanism 24, the straps 18, 20 cannot be pulled back out of the apertures 14, 16. Therefore, once engaged, the straps 18, 20 can only be tightened.

The restraint device 10 further includes one or more sensors 26 for sensing at least one parameter associated with the wearer of the restraint device 10. In the embodiment shown, a sensor 26 is positioned on an inside portion of at least one of the straps 18, 20. In this configuration, the sensor 26 may contact the skin of a wearer, such that the sensor 26 is suitable to detect a physiological parameter associated with the wearer, such as, but not limited to, skin temperature, skin pH, heart rate, the level of blood-oxygen saturation, a virulent bacterial status, or any other physiological parameter of the wearer. In one embodiment, the sensor 26 may incorporate nanotechnology devices or structures to enable the sensor 26 to detect a virulent bacterial status of a wearer, such as, but not limited to Methicillin-resistant Staphylococcus aureus (MRSA), Beta Streptococcus, or other conditions of a wearer of the restraint device 10.

Additionally, or alternatively, the sensor 26 may be configured to sense the presence of, or the exposure of the wearer to, at least one of a biological substance, a chemical substance, or a radioactive substance. For example, a biological or chemical substance may be associated with an explosive material, a chemical weapon, or a dangerous biological substance. In other embodiments, the sensor 26 may alternatively, or additionally, be configured to detect the presence of or the exposure of the wearer to radioactive materials, illegal drugs, or other substances.

In accordance with another aspect, the restraint device 10 may be configured to indicate when a particular parameter associated with the wearer of the restraint device 10 has been detected. Indication of the detected parameter may be a visual indication, an audible indication, or a combination thereof. Alternatively, or in addition, the restraint device 10 may be configured to store and/or transmit information related to the sensed parameter. Indication of the detected parameter and/or storage or transmission of information related to a sensed parameter may be accomplished by the sensor 26, itself, or may be provided for by, or in combination with, other components of the restraint device 10. In accordance with another aspect, the sensor 26 may be a nanotechnology enabled sensor capable of detecting the one or more sensed parameters.

In the embodiment shown, the restraint device 10 further includes a device 28 for receiving signals from the one or more sensors 26. The device 28 may be configured as a data storage device that is operable to store the information related to the one or more sensed parameters associated with the wearer, that stored data may be later obtained, communicated, or transmitted for use by a medical or law enforcement professional, for example.

In the embodiment shown, device 28 includes a restraint RFID device 29 that may be configured as a transmitter, operable to transmit information associated with the one or more sensed parameters. For example, the restraint RFID device 29 may transfer the information associated with the one or more sensed parameters via Radio Frequency Identification (RFID) communication. In another example, the restraint RFID device 29 may transfer the information associated with the one or more sensed parameters via Near Field Communication (NFC).

The information associated with the one or more sensed parameters may be transmitted to a proximate communications device, such as, but not limited to a cellular phone (not shown), that is in possession of personnel proximate to the wearer of the restraint device 10. The proximate communications device may be located within proximity of the restraint RFID device 29 so that the proximate communications device may receive the transmitted information associated with the one or more sensed parameters from the restraint RFID device 29. For example, the proximate communications device may be within 20 cm of the restraint RFID device 29 to adequately receive the transmitted information associated with the one or more sensed parameters from the restraint RFID device 29 using NFC.

The stored or transmitted information by the restraint RFID device 29 may include the sensed parameters themselves, or may be related to the sensed parameters. The restraint RFID device 29 may select which information associated with the sensed parameters to transmit to the proximate communications device. As a result, information that is transmitted from the restraint RFID device 29 that is associated with sensed parameters may be filtered by the restraint RFID device 29 so that any unauthorized information associated with the sensor parameters may not be shared with the proximate communications device.

As a non-limiting example, the restraint RFID device 29 may transmit the information associated with a sensed parameter that may include an alarm indicating that a particular condition exists (or has ceased to exist), or that a particular threshold of sensed parameters has been traversed. The restraint RFID device 29 may transmit the information continuously or periodically. In an exemplary embodiment, the restraint RFID device 29 may communicate the information associated with the sensed parameter of a wearer to the proximate communications device that is in possession of attendant personnel, such as medical or law enforcement professionals, whereby the attendant in possession of the proximate communications device may take immediate action as necessary. In other applications, the information transmitted from the restraint RFID device 29 to the proximate communications device may be collected and analyzed with information from other sources, such as information from other restrained individuals, for research, law enforcement, and/or various other purposes.

In an embodiment, the device 28 may also include a rectenna (not shown). The rectenna is an antenna that may convert the RF and/or NFC energy generated by the RFID device 29 to transfer the information associated with the one or more sensed parameters to the proximate communications device to power the device 28, RFID device 29, and/or the sensor 26. The rectenna may convert the RF and/or NFC energy into alternating current (AC) induced in the rectenna by the electromagnetic waves included in the RF and/or NFC energy. The rectenna may then generate direct current (DC) power from the AC to power the device 28, RFID device 29, and/or the sensor 26.

While the restraint device 10 depicted in FIG. 1 is shown to include a single sensor 26 positioned for contact with the skin of a wearer of the restraint device 10, it will be appreciated that more than one sensor 26 may alternatively be used, and that the one or more sensors 26 may alternatively, or additionally, be placed on other portions of the restraint device 10, as may be desired. For example, when it is desired to sense a parameter that does not necessarily require contact between a sensor 26 and the skin of a wearer, the sensor 26 may be placed at other suitable positions on the restraint device 10. It will also be appreciated that one or more sensors 26 may be incorporated directly into the restraint device 10 during manufacture of the restraint device 10, or that one or more sensors 26 may be added to a pre-existing restraint device to obtain a restraint device 10 in accordance with the principles of the present disclosure. In accordance with another aspect, the sensor 26 may be a nanotechnology enabled sensor capable of detecting the one or more sensed parameters.

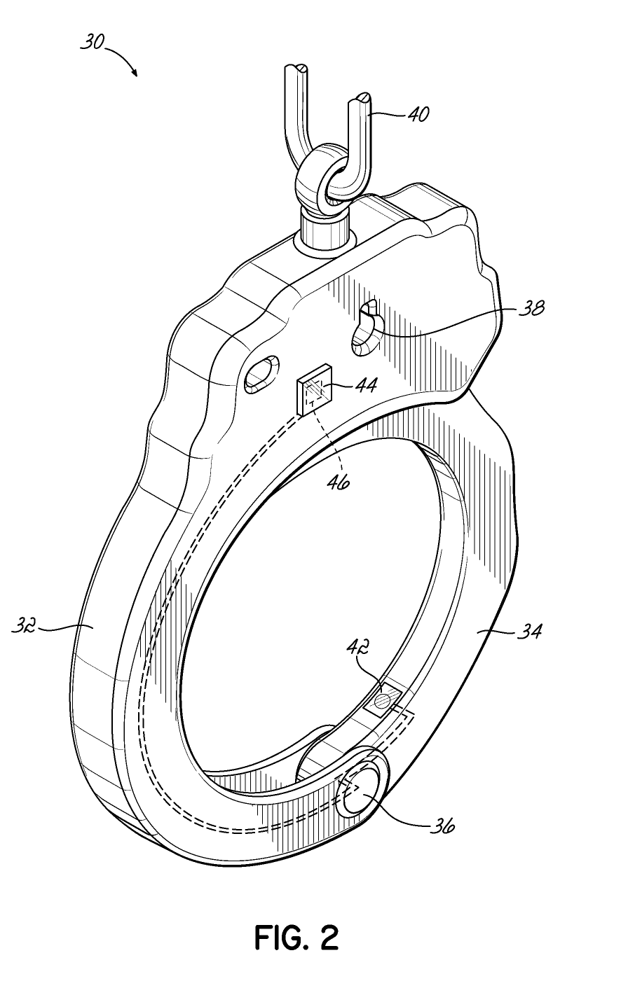

While the restraint device 10 depicted in FIG. 1 is generally a disposable type restraint, FIG. 2 depicts another exemplary embodiment of a restraint device in accordance with the principles of the present disclosure in the form of handcuffs 30, which may constitute a form of a reusable restraint device. In the embodiment shown, the handcuffs 30 are of a conventional handcuff construction, including a cheek plate assembly 32 and an adjustable bow 34 pivotally coupled to the cheek plate assembly 32 by a pivot joint 36. While not specifically illustrated herein, the bow 34 includes a plurality of gear teeth that are engaged by a locking mechanism within the cheek plate assembly 32 and which are releasable by manipulation of an appropriate key inserted into a keyway 38. While the embodiment shown in FIG. 2 depicts only a single cuff, it will be appreciated that a pair of cuffs may be coupled together, such as by chain links 40, as known in the art.

With continued reference to FIG. 2, the handcuffs 30 include at least one sensor 42 positioned on the handcuffs 30 for sensing a desired parameter associated with the wearer of the handcuffs 30. In this embodiment, a sensor 42 is positioned on an inwardly facing portion of the bow 34 for contact with the skin of a wearer of the handcuffs 30. It will be appreciated, however, that more than one sensor 42 may be used with the handcuffs 30, and that the sensors 42 may be positioned on various other portions of the handcuffs 30, as may be desired. As discussed above, the sensor 42 may be configured to sense a physiological parameter associated with the wearer, such as a virulent bacterial status of the wearer, a skin temperature, a skin pH, a heart rate, or a blood-oxygen saturation of the wearer. Alternatively, or in addition, the sensor 42 may be configured to sense the presence of, or the exposure of the wearer to, at least one of a biological substance, a chemical substance, or a radioactive substance. In accordance with another aspect, the sensor 42 may be a nanotechnology enabled sensor capable of detecting the one or more sensed parameters.

The handcuffs 30 may be further configured to indicate detection of the sensed parameter, such as by audio, visual, or other methods. Indication of a detected parameter may be accomplished by the sensor 42, itself, or indication may be provided for by, or in combination with, additional components of the handcuffs 30. In the embodiment shown, the handcuffs 30 include a device 44 for receiving signals from the one or more sensors 42. The device 44 may be configured as a data storage device operable to store information associated the sensed parameter of the wearer. The device 44 may include a restraint RFID device 46 that may be configured as a transmitter operable to transmit information associated with the sensed parameter to a proximate communications device, as discussed above. For example, the restraint RFID device 46 may transfer the information associated with the one or more sensed parameters via RFID communication. In another example, the restraint RFID device 46 may transfer the information associated with the one or more sensed parameters via NFC.

In an embodiment, the device 44 may also include a rectenna (not shown). The rectenna may convert the RF and/or NFC energy generated by the RFID device 46 to transfer the information associated with the one or more sensed parameters to the proximate communications device to power the device 44, RFID device 46, and/or the sensor 42. The rectenna may convert the RF and/or NFC energy into alternating current (AC) induced in the rectenna by the electromagnetic waves included in the RF and/or NFC energy. The rectenna may then generate direct current (DC) power from the AC to power the device 44, RFID device 46, and/or the sensor 42.

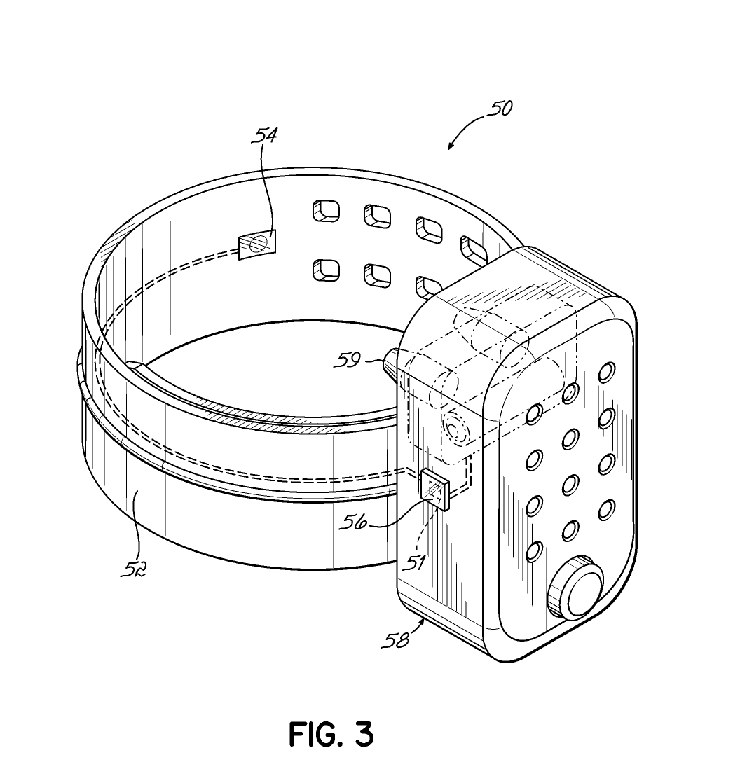

While the restraint devices 10, 30 depicted in FIGS. 1 and 2 illustrate embodiments configured to restrain movement of at least a first body portion of a wearer relative to a second body portion of the wearer, FIG. 3 illustrates another exemplary embodiment of a restraint device in accordance with the principles of the present disclosure wherein the device is configured to limit movement of at least a first body portion of the wearer with respect to a defined location or space. In this embodiment, the restraint device is in the form of a single cuff 50 that may be placed upon the ankle, wrist, or other suitable portion of an individual's body. To this end, the cuff 50 includes an elongate band 52 that may be placed around a portion of the wearer's body, such as an ankle. With the band 50 fitted securely to the wearer, the band 52 may be locked in place so that it cannot easily be removed by the wearer. The cuff 50 further includes one or more sensors 54 positioned on the cuff 50 to sense a desired parameter associated with the wearer of the cuff 50. In the embodiment shown, a sensor 54 is positioned on a portion of the band 52 for contact with the skin of the wearer. It will be appreciated, however, that the one or more sensors 54 may be positioned on various other portions of the cuff 50, as may be desired.

As discussed above, the sensor 54 may be configured to sense a physiological parameter associated with the wearer, such as a virulent bacterial status of the wearer, a skin temperature, a skin pH, a heart rate, or a blood-oxygen saturation of the wearer. Alternatively, or in addition, the sensor 54 may be configured to sense the presence of, or the exposure of the wearer to, at least one of a biological substance, a chemical substance, or a radioactive substance. The cuff 50 may be further configured to indicate detection of the sensed parameter, such as by audio, visual, or other methods. Indication of a detected parameter may be accomplished by the sensor 54, itself, or indication may be provided for by, or in combination with, additional components of the cuff 50. In accordance with another aspect, the sensor 54 may be a nanotechnology enabled sensor capable of detecting the one or more sensed parameters.

In the embodiment shown, the cuff 50 further includes a device 56 for receiving signals from the one or more sensors 54. The device 56 may be configured as a data storage device operable to store information associated with the sensed parameter of the wearer. The device 56 may include a restraint RFID device 51 that may be configured as a transmitter operable to transmit information associated with the sensed parameter to a proximate communications device as, discussed above. In this embodiment, the cuff 50 may be configured to transmit signals containing information, such as the location of the wearer of the cuff 50 and/or the one or more sensed parameters associated with the wearer of the cuff 50. If the wearer moves outside of a permitted space, signals may be transmitted to the proximate communications device to notify an appropriate monitoring authority. For example, the restraint RFID device 51 may transfer the location and/or the one or more sensed parameters associated with the wearer of the cuff 50 via RFID communication. In another example, the restraint RFID device 51 may transfer the location and/or the one or more sensed parameters associated with the wearer of the cuff 50 via NFC. Alternatively, or in addition, the cuff 50 may be configured to emit an audible sound to indicate that the wearer of the cuff 50 has moved outside the permitted space.

In another embodiment, the cuff 50 may be configured to apply a disabling electric shock to incapacitate the wearer of the cuff 50 if an attempt is made to leave the permitted space. In such an embodiment, the cuff 50 may include a module 58 having electronic circuitry including a power source, one or more transformers, one or more capacitors, and other components capable of generating an electric charge and applying the electric charge to the wearer via one or more electrodes 59 positioned at an appropriate location on the cuff 50. The module 58 may also provide power to the device 56, the restraint RFID device 51, and/or the sensor 54.

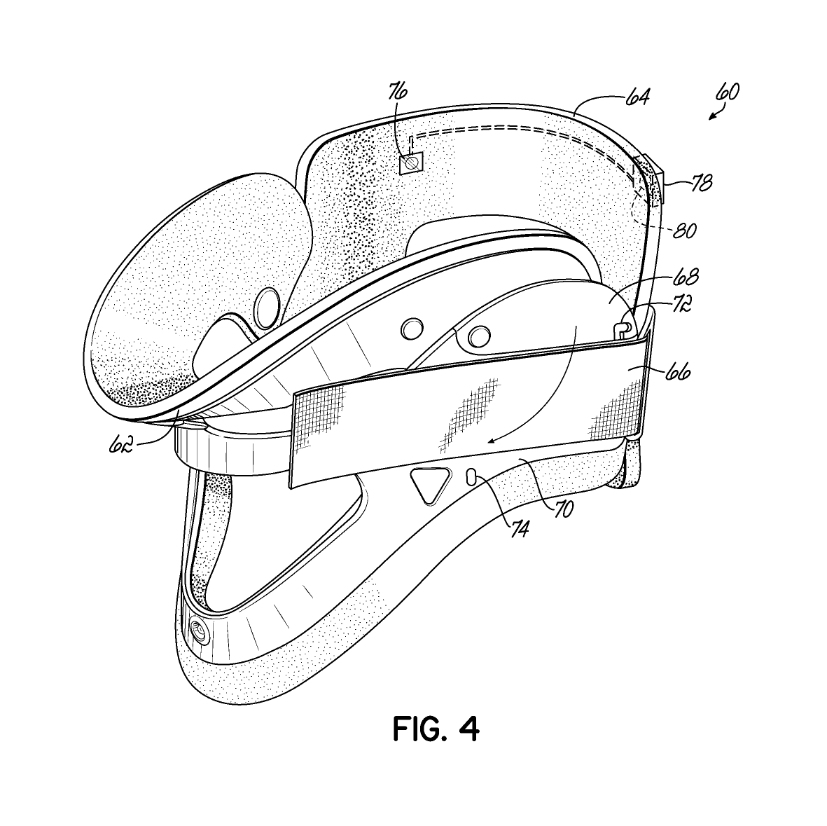

FIG. 4 depicts another exemplary restraint device in accordance with the principles of the present disclosure, in the form of a cervical collar 60. The cervical collar 60 depicted in FIG. 4 is similar to the cervical collar generally shown and described in U.S. Pat. No. 7,883,485, the disclosure of which is incorporated by reference herein in its entirety. The cervical collar 60 includes first and second support portions 62, 64 configured to immobilize, or at least brace the patient's head and neck region with respect to the rest of the wearer's body. In this embodiment, the first and second support portions 62, 64 may be arranged into a generally annular configuration and secured with a connector strap 66, such as a hook-and-loop type fastening strap system, for example. The cervical collar 60 further includes a pivoting lever arm 68 secured to a plastic piece 70 and pivotally moveable over the connector strap 66 after the cervical collar 60 has been fitted around the neck of the wearer. A slot or recess 72 formed into the pivoting lever arm 68 may be engaged with a corresponding latch 74 to contain the connector strap 66 and thereby prevent removal of the connector strap 66 from the first support portion 62 of the cervical collar 60 until the lever arm 68 is unlocked and pivoted out of the way, as generally described in U.S. Pat. No. 7,883,485.

The cervical collar 60 further includes one or more sensors 76 disposed on a portion of the collar 60 to sense one or more parameters associated with the wearer of the collar 60. In the embodiment shown, a sensor 76 is provided on an interior portion of the collar 60 at a location for contacting the skin of the wearer. It will be appreciated that more than one sensors 76 may alternatively be provided, and that such sensors 76 may alternatively, or additionally, be positioned on any suitable surface of the collar 60 to sense a desired a parameter. The one or more sensors 76 may be configured to sense a physiological parameter associated with the wearer, such as a virulent bacterial status of the wearer, a skin temperature, a skin pH, a heart rate, or a blood-oxygen saturation of the wearer. Alternatively, or in addition, the sensors 76 may be configured to sense the presence of, or the exposure of the wearer to, at least one of a biological substance, a chemical substance, or a radioactive substance. In accordance with another aspect, the sensor 76 may be a nanotechnology enabled sensor capable of detecting the one or more sensed parameters.

The cervical collar 60 may be further configured to indicate detection of the sensed parameter, such as by audio, visual, or other methods. Indication of a detected parameter may be accomplished by the sensor 76, itself, or indication may be provided for by, or in combination with, additional components of the cervical collar 60. In the embodiment shown, the cervical collar 60 further includes a device 78 for receiving signals from the one or more sensors 76. The device 78 may be configured as a data storage device operable to store information related to the sensed parameter of the wearer. The device 78 may include a restraint RFID device 80 that may be configured as a transmitter operable to transmit information related to the sensed parameter to a proximate communications device. For example, the restraint RFID device 80 may transfer the information associated with the one or more sensed parameters via RFID communication. In another example, the restraint RFID device 80 may transfer the information associated with the one or more sensed parameters via NFC.

In an embodiment, the device 78 may also include a rectenna (not shown). The rectenna is an antenna that may convert the RF and/or NFC energy generated by the restraint RFID device 80 to transfer the information associated with the one or more sensed parameters to the proximate communications device to power the device 78, the restraint RFID device 80, and/or the sensor 76. The rectenna may convert the RF and/or NFC energy into alternating current (AC) induced in the rectenna by the electromagnetic waves included in the RF and/or NFC energy. The rectenna may then generate direct current (DC) power from the AC to power the device 78, the restraint RFID device 80, and/or the sensor 76.

In use, a restraint device, such as, but not limited to, any of the restraint devices 10, 30, 50, 60 described herein, may be placed on at least a portion of an individual's body such that the restraint device 10, 30, 50, 60 restrains the movement of at least the portion of the individual's body. The restraint device 10, 30, 50, 60 may restrain movement of at least the portion of the individual's body with respect to another portion of the individual's body, or the restraint device 10, 30, 50, 60 may restrain movement with respect to a permitted space.

Any of the restraint devices 10, 30, 50, 60 described herein may restrain a portion of the individual's body with respect to another portion of the individual's body or a stationary object position within proximity of the individual's body via an electronic connection between the portion of the individual's body and the other portion of the individual's body or the stationary object. For example, any of the restraint devices 10, 30, 50, 60 described herein may be a bracelet that an individual wears on his/her wrist. The individual may be restrained to his/her bed by an electronic connection between the bracelet worn on the individual's wrist and a sensor coupled to the individual's bed. Appropriate personnel may be alerted when the bracelet worn by the individual moves beyond a specified distance from the sensor coupled to the bed, thus breaking the electronic connection between the bracelet worn by the individual and the bed.

Any of the restraint devices 10, 30, 50, 60 described herein may restrain a portion of the individual's body with respect to another portion of the individual's body or a stationary object position within proximity of the individual's body via a communication connection between the portion of the individual's body and the other portion of the individual's body or the stationary object. For example, the communication connection may be between the bracelet worn on the individual's wrist and a communication station programmed to communicate with the bracelet. The individual may be restrained to a region where the communication between the bracelet and the communication station may be maintained. The communication station may be base station, micro cell, pico cell, femtocell, Wi-Fi station, and/or any other communication station that may maintain communication with the bracelet when the bracelet is within the boundaries of the communication station that will be apparent to those skilled in the relevant art(s) without departing from the spirit and scope of the invention. The individual may be limited to a region of communication provided by a specified communication station. The individual may also be limited to a combination of regions of communication provided by a combination of communication stations.

Any of the restraint devices 10, 30, 50, 60 described herein may restrain a portion of the individual's body with respect to another portion of the individual's body or a stationary object position within proximity of a proximate communications device that is within proximity of the restraint devices 10, 30, 50, 60. The proximate communications device is within proximity of the restraint devices 10, 30, 50, 60 when the information associated with the one or more sensed parameters is adequately transmitted from the restraint devices 10, 30, 50, 60 to the proximate communications device as will be discussed in more detail below. For example, the communication connection may be between the bracelet worn on the individual's wrist and the proximate communications device. The individual may be restrained to where the communication between the bracelet and the proximate communications device may be maintained.

Any of the restraint devices 10, 30, 50, 60 may also restrain a portion of an object in a similar fashion as restraining a portion of the individual's body as described in detail above and below. Any of the restraint devices 10, 30, 50, 60 may also restrain a portion of an animal in a similar fashion as restraining a portion of the individual's body as described in detail above and below. The restraint device 10, 30, 50, 60 includes at least one sensor 26, 42, 54, 76 as described above, whereby the at least one sensor 26, 42, 54, 76 senses at least one parameter associated with the wearer.

Once the parameter is sensed, the restraint device 10, 30, 50, 60 may be configured to indicate that the sensed parameter has been detected so that appropriate action may be taken. Information related to the sensed parameter may be stored or may be transmitted to appropriate personnel in possession of a proximate communications device, such as medical or law enforcement personnel, which may be located proximate to the wearer of the restraint device 10, 30, 50, 60.

In another aspect in accordance with the principles of the present invention, a restraint device 10, 30, 50, 60, such as those described above, may be configured to provide visual indication of the detection of a sensed parameter in dark or low-light environments. For example, the sensor 26, 42, 54, 76, the restraint RFID device 29, 46, 51, 80, or other components of the restraint device 10, 30, 50, 60 may be configured to illuminate upon the detection of a sensed parameter. Illumination may be provided by phosphorescent materials, electrical components, or any other suitable components or features capable of providing or facilitating visual indication in dark or low-light conditions. Moreover, the restraint devices themselves may be formed from or with phosphorescent or other luminous materials to facilitate visibility of the restraint device 10, 30, 50, 60 in dark or low-light conditions.

Within the context of this patent the purpose of a sensor is to monitor the environment surrounding a restraint device. This monitoring would include measuring properties both directly and indirectly of a number of sensors.

Examples of such sensors would include but not be limited to:

1. Signal transducers such as: photoelectric, photodielectric, photoluminescence, electroluminescence, and chemiluminescence, thermoelectric.

2. Transduction platforms such as: optical waveguide based transducers (optical fiber, interferometric optical transducers or surface plasmon resonance transducers).

3. Conductometric and capacitive transducers (nanostructured SnO₂ thin film exposed to O₂ gas at 400° C.).

4. Electrochemical transducers (electrochemical pH sensors).

5. Solid state transducers (p-n diodes or bipolar junction based transducers or Schottky diode based or MOS based).

6. Inorganic nanotechnology enabled sensors (gas sensing nanostructured thin films, phonons, nano-mechanical sensors, nano enabled optical sensors, nano enabled magnetically engineered spintronic sensors).

7. Organic nanotechnology enabled sensors such as proteins, antibodies, enzymes, etc. Nano-sensors based on nucleotides and DNA.

8. Acoustic wave transducers such as film bulk acoustic wave resonator, cantilever based transducers or inerdigitally launched surface acoustic wave devices (SAW).

The sensor 26, 42, 54, 76 as described above, may be a nanotechnology enabled sensor capable of detecting the one or more sensed parameters. The nanotechnology enabled sensors may include a nanosensor array with enhanced sensing capabilities in that the nanosensor array may capture particles that are associated with the sensor parameters so that the sensor parameters may be identified, stored, and/or transmitted. For example, the nanotechnology enabled sensor that includes the nanosensor array is placed on an individual who has recently handled explosive materials. The nanosensor array detects particles associated with the explosive materials. The information associated with the detected particles may then be identified, stored, and/or transmitted as the sensor parameters.

As noted above, the sensor parameters captured by the nanotechnology enabled sensor 26, 42, 54, 76 may be provided to the restraint RFID device 29, 46, 51, 80 for transmission. The restraint RFID device 29, 46 51, 80 may be coupled to the nanotechnology enabled sensor 26, 42, 54, 76 so that the restraint RFID device 29, 46, 51, 80 may receive the sensor parameters from the nanotechnology enabled sensor 26, 42, 54, 76 and transmit information associated with the sensor parameters to a proximate communications device.

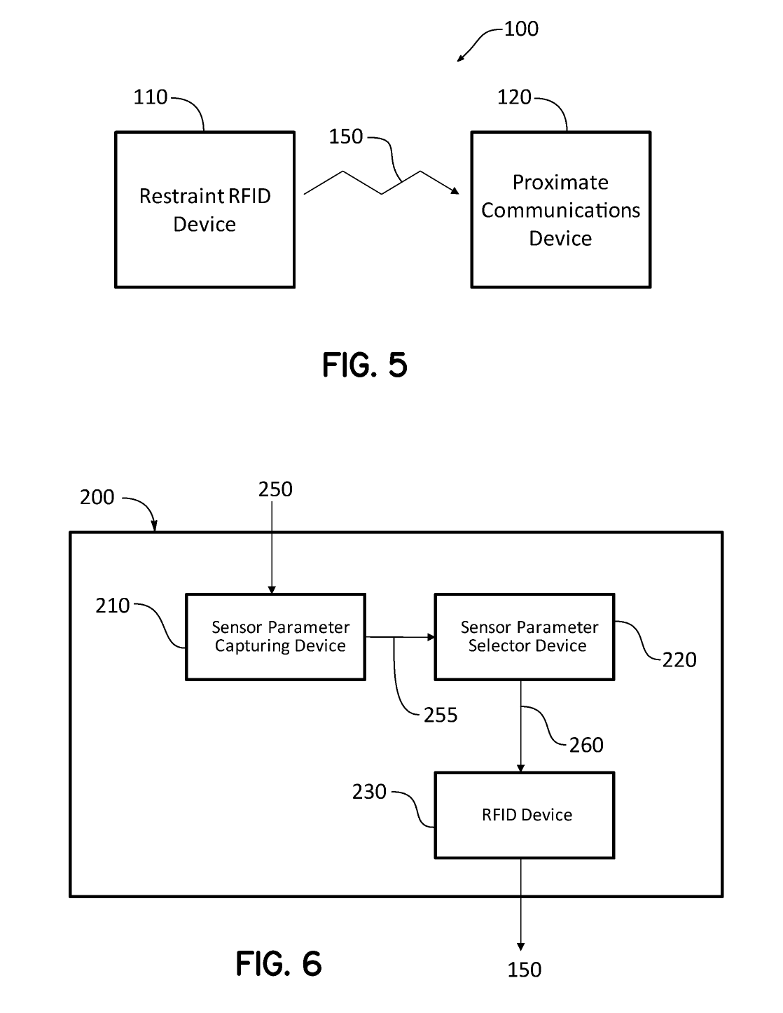

FIG. 5 illustrates a block diagram of a communication configuration according to an exemplary embodiment of the invention. A restraint RFID device 110 and a proximate communications device 120 may be engaged within a communication configuration 100. Examples of the restraint RFID device 110 may include but not limited to restraint RFID device 29, 46, 51, 80. The restraint RFID device 110 may be coupled to a restraint device such as but not limited restraint devices 10, 30, 50, 60, for example. The restraint RFID device 110 may receive and store information associated with the one or more sensor parameters received from a sensor such as but not limited to sensor 26, 42, 56, 76, for example. The restraint RFID device 110 may transmit the information associated with the one or more sensor parameters as transmitted sensor parameters 150 to the proximate communications device 120 via RFID communication. The proximate communications device 120 may receive the transmitted sensor parameters 150 from the restraint RFID device 110 and process the transmitted sensor parameters 150.

As noted above, the sensor parameters provided to the restraint RFID device 110 may be based on particles detected by nanotechnology enabled sensors. The transfer of the transmitted sensor parameters 150 from the restraint RFID device 110 to the proximate communications device 120 may occur via RFID communication.

An example of RFID communication that the restraint RFID device 110 may communicate the transmitted sensor parameters 150 may include NFC. NFC is a set of standards to establish radio communication between the restraint RFID device 110 and the proximity communications device 120. NFC may allow the restraint RFID device 110 to communicate the transmitted sensor parameters 150 to the proximate communications device 120 when the restraint RFID device 110 is within proximity of the proximity communications device 120. The restraint RFID device 110 may be within proximity of the communications device 120 to appropriately communicate the transmitted sensor parameters 150 when the restraint RFID device 110 is within 20 cm of the proximity communications device 120. The NFC communication of the transmitted sensor parameters 150 may occur with but not limited to Type A NFC, Type B NFC, and/or FeliCa NFC.

NFC uses magnetic induction between two loop antennas located within each other's near field to form an air core transformer. The restraint RFID device 110 may be active or passive when engaging in NFC with the proximate communications device 120. In an embodiment, the restraint RFID device 110 may be passive in that the restraint RFID device 110 is a target as defined by the NFC protocol. The restraint RFID device 110 may include a NFC tag that contains the transmitted sensor parameters 150 that the proximate communications device 120 may read. The NFC tag may include information associated with the sensor parameters that may include but not limited to physiological parameters retrieved from the wearers of the cervical restraints, posey restraints, and/or handcuffs. The NFC tag may include an initiator device that provides a carrier field. The restraint RFID device 110 may receive its power from an electromagnetic field provided by an initiator as defined in the NFC protocol

The proximate communications device 120 may be the initiator in the NFC protocol in that the proximate communications device 120 may generate its own power to engage the restraint RFID device 110 in NFC and receive the transmitted sensor parameters 150 from the restraint RFID device 110. The proximate communications device 120 may read, collect, store, and/or transmit the transmitted sensor parameters 150 to an end user. The end user may include an individual that is holding the proximate communications device 120 within proximity of the restraint RFID device 110 so that the transmitted sensor parameters 150 may be adequately communicated from the restraint RFID device 110 to the proximate communications device 120 via NFC. For example, the end user may include but is not limited to a nurse, and/or a law enforcement officer.

The restraint RFID device 110 may transmit the transmitted sensor parameters 150 to the proximate communications device 120 with minimum power when passively communicating the transmitted sensor parameters 150 via NFC. The NFC of the transmitted sensor parameters 150 may be open, standards based, secure and compatible with existing cellular phone technology such as but not limited to Nokia 6131 NFC and C7, SAGEM's my 700x, Samsung's D500E and Galaxy S II, BenQ's T80, Sagem's Cosyphone, Google's Nexus S, RIM Blackberry Bold 9900 and 9930.

The restraint RFID device 110 may transmit the transmitted sensor parameters 150 to the proximate communications device 120 via other wireless communications standards that include but are not limited to Bluetooth 3.0, Bluetooth 2.1, Wi-Fi B, Wi-Fi G, Wi-Fi N, and/or ZigBee®. The restraint device 110 may transmit the sensor parameters 150 to the proximate communications device 120 via international standards that include but are not limited to IEEE 802.15.1, ISO 14443, IEEE802.11b, IEEE802.11g, IEEE802.11n, and/or IEEE802.15.4. The restraint device 110 may transmit the transmitted sensor parameters 150 to the proximate communications device 120 via frequency ranges that include but are not limited to 13.56 MHz to 2.4 GHz, 5 GHz, and/or terahertz frequencies. The restraint RFID device 110 may transmit the transmitted sensor parameters to the proximate communications device 120 at data rate ranges that include but are not limited to 0.25 Mb/s to 54 Mb/s. The restraint RFID device 110 may transmit the transmitted sensor parameters 150 to the proximate communications device 120 when within a range of each other that includes but not is not limited to 0-20 cm for NFC communication, 0-100 m for Bluetooth communication, and/or 100-1200 m for ZigBee® communication.

The restraint RFID device 110 may encrypt the transmitted sensor parameters 150 before transmitting the transmitted sensor parameters 150 to the proximate communications device 120. The restraint RFID device 110 may encrypt the transmitted sensor parameters 150 to prevent any unauthorized individual and/or computing device from obtaining the transmitted sensor parameters 150 so that the providing of the transmitted sensor parameters 150 is limited between the restraint RFID device 110 and the proximate communications device 120. The proximate communications device 120 may then decrypt the transmitted sensor parameters 150 so that the proximate communications device 120 may analyze the transmitted sensor parameters 150 for a user of the proximate communications device 120 to take any action necessary. The transmitted sensor parameters 150 may be encrypted and/or decrypted using any encryption and/or decryption method to adequately prevent unauthorized individuals and/or computing devices from intercepting the transmitted sensor parameters 150 that will be apparent to those skilled in the relevant art(s) without departing from the spirit and scope of the invention.

FIG. 6 illustrates a block diagram of a restraint RFID device according to an exemplary embodiment of the invention. The restraint RFID device 200 intelligently routes information associated with the sensor parameters captured from a sensor to a proximate communications device based upon transmitted sensor parameters selected from the information associated with the sensor parameters. The restraint RFID device 200 utilizes a RFID device to send the transmitted sensor parameters to the proximate communications device. The restraint RFID device 200 includes a sensor parameter capturing device 210, a sensor parameter selector device 220, and an RFID device 230. The restraint RFID device 200 includes many similar features with the restraint RFID device 110; therefore, only the differences between the restraint RFID device 110 and the restraint RFID device 200 are to be discussed in further detail.

The sensor parameter capturing device 210 may capture the sensor parameter information 250 from a sensor coupled to a restraint device. For example the sensor capturing device 210 may capture the sensor parameter information 250 from sensor 26, 42, 54, 76 coupled to restraint device 10, 30, 50, 60. As noted above, the sensor 26, 42, 54, 76 may be nanotechnology enabled sensors. The sensor parameter information 250 may be the information associated with the sensor parameters captured by the sensors 26, 42, 54, 76. Sensor parameter capturing device 210 may provide the captured sensor parameter information 255 to sensor parameter selector device 220.

Sensor parameter selector device 220 may select the selected sensor parameter information 260 from the captured sensor parameter information 255. The selected sensor parameter information 260 may include selected information associated with the sensor parameters that is to be transmitted to the proximate communications device 120. In an embodiment, a portion of the captured sensor parameter information 255 may not be authorized to be transmitted to the proximate communications device 120. For example, a portion of the captured sensor parameter information 255 may include private information associated with the wearer of the restraint device 10, 30 50, 60 that is not authorized by the wearer to be transmitted to the proximate communications device 120. Thus, the sensor parameter selector device 220 selects a portion of the captured sensor parameter information 255 that is authorized to be transmitted to the proximate communications device 120. The sensor parameter selector device 220 provides the authorized portion of the sensor parameter information 255 to the RFID device 230 as authorized sensor parameter information 260.

The RFID device 230 may transmit the authorized sensor parameter information 260 to the proximate communications device 255 as the transmitted sensor parameters 150. As shown in FIG. 6, the RFID device 230 modulates information onto the transmitted sensor parameters 150 from the authorized sensor parameter information 260 and transmits the transmitted sensor parameters to the proximate communications device 120. When the RFID device 230 is operating in the passive communication mode, the RFID device 230 modulates the information onto the magnetic field that is generated by the proximate communications device 120. Otherwise, the RFID device 230 generates its own magnetic field and modulates the information onto its own magnetic field when the RFID device 230 is operating in the active communication mode. The RFID device 230 upconverts, modulates, and/or encodes the transmitted sensor parameters 150 to transmit information, such as one or more sensor parameters to the proximate communications device 120 for example, that are embedded within the transmitted sensor parameters 150.

In an embodiment, the restraint RFID device 200 may include a fully roll to roll gravure printed rectenna on plastic foils for wireless power transmission at 13.56 MHz. The restraint RFID device 200 may require DC power of less than 0.3 W provided by inductively coupled AC from a 13.56 MHz power transmitter through the rectenna. The rectenna may include an antenna, a diode, and a capacitor which is printed via a roll to roll gravure printing process using plastic foils. The restraint RFID device 200 may use four different nanoparticle based inks to print the rectenna that includes the antenna, a diode and a capacitor on plastic foils.

In another embodiment, the restraint RFID device 200 may include a wireless passive antenna printed on a silk substrate which has the ability to identify analytes and communicate data received from the analytes over multiple regions of the electromagnetic spectrum. Such wireless passive antennas may be easily applied to curved structures that include but are not limited to a cervical collar, dog collar, seat belt, handcuff, a shipping security web, and/or an airport body scanner and used to identify analyte priorities.

In another embodiment, the restraint RFID device 200 may include an antenna or an antenna array of antennas/resonators made of sub-micron thickness of gold. The LC resonators may have behaviors that are dependent on the geometry and dielectric properties of the material. Any change in the electromagnetic properties, such as but not limited to resistivity, capacitance, and/or inductance for example, of the antenna may result in a change in the resonant response resulting in the LC resonators being sensitive to the local environment. The capturing of changes in the electromagnetic properties provide restraint RFID device 200 the capability of analyzing analytes in a local environment of the restraint device 10, 30 50, 60.

In an embodiment, the nanotechnology enabled sensors 26, 42, 54, 76 may include graphene films printed onto a surface such as silk and combined with biomaterials resulting in a biointerfaced sensing platform that may be selectively tuned to detect bacteria at a cellular level.

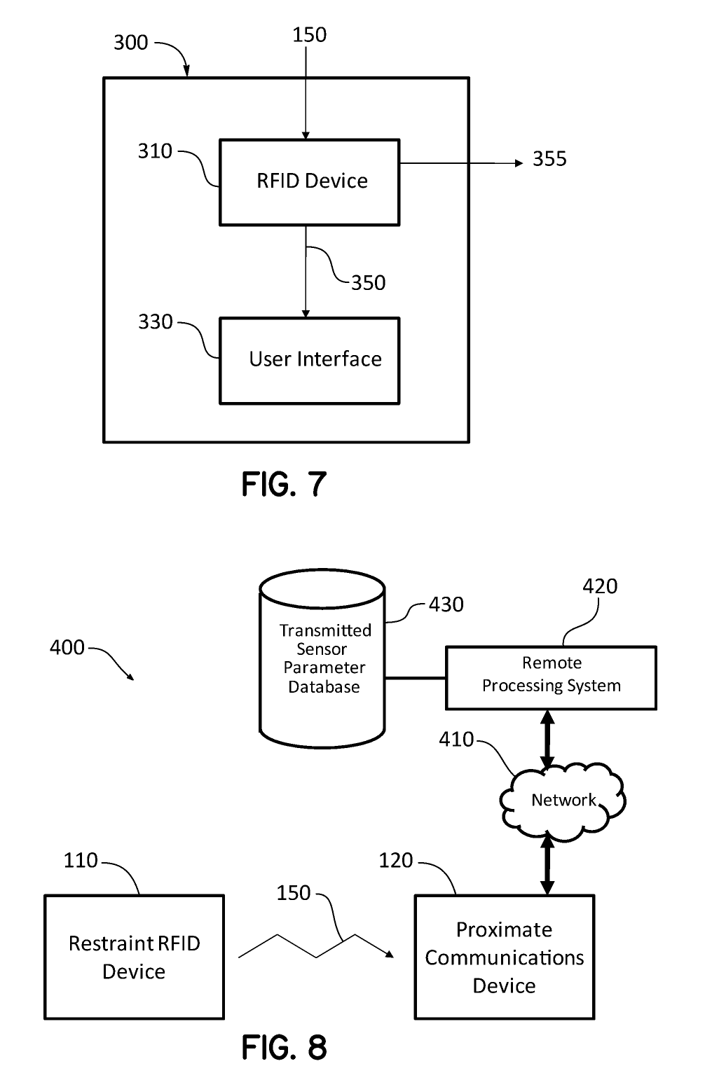

FIG. 7 illustrates a block diagram of a proximate communications device according to an exemplary embodiment of the invention. A RFID device may be integrated within the proximate communications device 300. The RFID device may route the transmitted sensor parameters to a user interface for analysis by an end user in possession of the proximate communications device 300. The RFID device may also route the transmitted sensor parameters to a remote location for processing. The proximate communications device 300 includes a RFID device 310 and a user interface 330. The proximate communications device 300 includes many similar features with the proximate communications device 120; therefore, only the differences between the proximate communications device 120 and the proximate communications device 300 are to be discussed in further detail.

The proximate communications device 300 may represent a mobile telephone, a portable computing device, other computing devices such as a personal computer, a laptop, or a desktop computer, and/or any other suitable electronic device that can be used within proximity of the restraint RFID device 110 coupled to restraint 10, 30, 50, 60 to receive the transmitted sensor parameters 150 from the restraint RFID device 110 that will be apparent to those skilled in the relevant art(s) without departing from the spirit and scope of the invention.

As discussed above, the proximate communications device 300 may be integrated with the RFID device 310. The RFID device 310 interacts with the restraint RFID device 110 to exchange information, such as the transmitted sensor parameters 150 to provide an example, between the devices. The RFID device 310 may operate according to an active communication mode and/or a passive communication mode. In the active communication mode, the RFID device 310 generates a magnetic field and modulates this magnetic field with information to provide remote sensor parameters 355 and/or user interface sensor parameters 350. Otherwise, the RFID device 310 modulates information onto a magnetic field that is provided by another RFID capable device with the information to provide remote sensor parameters 355 and/or user interface sensor parameters 350 in the passive communication mode.

As shown in FIG. 7, the RFID device 310 recovers information from the transmitted sensor parameters 150 from the restraint RFID device 110. When the RFID device 310 is operating in the active communication mode, the restraint RFID device 110 modulates the information onto the magnetic field that is generated by the RFID device 310. Otherwise, the restraint RFID device 110 generates its own magnetic field and modulates this magnetic field with the information when the RFID device 310 is operating the passive communication mode.

The RFID device 310 downconverts, demodulates, and/or decodes the transmitted sensor parameters 150 to recover information, such as one or more sensor parameters for analysis by the end user for example, that are embedded within the received transmitted sensor parameters 150. The RFID device 310 provides this information to the user interface 330 as user interface sensor parameters 350 as the sensor parameters to be analyzed by the end user. The user interface 330 may display the user interface sensor parameters 350 to the end user for the user to analyze.

The RFID device 310 downconverts, demodulates, and/or decodes the transmitted sensor parameters 150 to recover information, such as one or more sensor parameters for analysis by the end user for example, that are embedded within the received transmitted sensor parameters 150. The RFID device 310 provides this information to a remote location as remote location sensor parameters 355 as the sensor parameters to be stored at a remote location and/or analyzed by end users located remotely from the proximate communications device 300.

FIG. 8 illustrates a block diagram of a transmitted sensor parameter configuration 400 according to an exemplary embodiment of the invention. The restraint RFID device 110 receives the transmitted sensor parameter information from the sensor 26, 42, 54, 76 and transmits the transmitted sensor parameter information 150 to the proximate communications device 120 via RFID communication. The processing communications device 120 may provide the transmitted sensor parameter information 150 via network 410 to a remote processing system 420. The remote processing system 420 may process the transmitted sensor parameter information 150 and/or store the transmitted sensor parameter information 150 in transmitted sensor parameter database 430.

For example, a patient is in a bed with a first vital sign nanosensor attached to an arm. The rail of the bed includes the restraint RFID device 110 and is communicating with the first vital sign nanosensor attached to the arm. The first vital sign nanosensor is electronically restrained, by distance, to the restraint RFID device 110. An alarm restraint may be activated when the electronic distance is exceeded. The first vital sign nanosensor may communicate physiological parameters to the restraint RFID device 110 when the electronic restraint is maintained. The restraint RFID device 110 may communicate the physiological parameters as the transmitted sensor parameters 150 to the proximity communications device 120 that is in possession of a nurse.

The nurse is able to download the physiological parameters into an EMR chart via the proximity communications device 120 in full compliance with Accountable Care Act. Security may be maintained via the proximate communications device 120 with a NFC security controller for the SIM card via the embedded secure element hardwired to the electronics of the proximate communications device 120. In addition, security may be maintained by a secure element that may be incorporated into a microSD card. This electronic restraint system that communicates with the electronic chart may save the nurse time from gathering physical vital signs.

The electronic chart may then be provided by proximate communications device 120 to remote processing system 420 via network 410 for remote processing. Remote processing system 420 may be a central processing system supported by a hospital network system. Remote processing system 420 may store the electronic chart in the transmitted sensor parameter database 430.

In another example, a driver is restrained by a seatbelt possession of the restraint RFID device 110 which is in communication with a nanosensor on the driver creating a bidirectional communication between the driver's nanosensor and the seatbelt restraint RFID device 110. The driver's nanosensor is able to fit adjacent to the driver's wrist and gather physiological parameters that are sent to the seatbelt restraint RFID device 110. The seatbelt restraint RFID device 110 then sends the physiological parameters as the transmitted sensor parameters 150 to the proximate communications device 120 which is in possession of the driver. The proximate communications device 120 then transmits the physiological parameters to remote processing system 420 for the car via network 410. The remote processing system 420 is able to monitor the physiological parameters of the driver. For example, the remote processing system 420 is able to monitor physiological parameters that include but are not limited to cardiac arrhythmias and/or oxygen levels to detect when oxygen levels may dip below a threshold that may be indicative of a stroke. The remote processing system 420 may be able to preemptively notify the driver that the driver is about to have a heart arrhythmia, to slow down the car, and/or call the medics. The remote processing system 420 may also store the physiological parameters in transmitted sensor parameter database 430 so that the driver's doctor may be able to obtain the physiological parameters for later medical examinations of the driver.

In another example, a person owns a dog that has a history of medical problems such as having a history of heart failure. A collar may be placed on the dog that includes nanosensors and the restraint RFID device 110. The restraint RFID device 110 may transmit vital physiological parameters of the dog as transmitted sensor parameters 150 to the proximate communications device 120 which is in possession of the dog owner. The proximate communications device 120 may provide the vital physiological parameters of the dog to the remote processing system 420 via the network 410. The remote processing system 420 may store the vital physiological parameters in the transmitted sensor parameter database 430.

In another example, an individual is diagnosed with a memory loss condition that includes but is not limited to autism, senile, and/or Alzheimer's. A piece of jewelry that may include but is not limited to a bracelet and/or ring may be placed on the individual that includes nanosensors and the restraint RFID device 110. The restraint RFID device 110 may transmit the location of the individual along with physiological parameters of the individual as transmitted sensor parameters 150 to the proximate communications device 120 which is in possession of a caregiver of the individual. The proximate communications device 120 may provide the location and the physiological parameters of the individual to the remote processing system 420 via network 410.

As a result, the caregiver may be notified by the proximate communications device 120 when the individual wanders beyond a threshold distance from the proximate communications device 120 which is in possession of the caregiver. The caregiver can then quickly retrieve the individual before the individual is harmed. The remote processing system may store the location and the physiological parameters in the transmitted sensor parameter database 430. A care giving facility, such as a nursing home for example, may monitor the movements of the individual and the physiological parameters based on such information stored in the transmitter sensor parameter database 430.

In another example, an individual suspected of plotting a terror operation that may cause harm to a large quantity of individuals and/or significant damage is detained before the terror operation is executed. Law enforcement places a restraint device that includes the nanotechnology capable sensors and the restraint RFID device 110 on the suspect during the detainment. The nanotechnology capable sensors capture particles that represent explosive materials from the suspect indicating that the suspect has recently been exposed to explosive materials. The nanotechnology capable sensors provide information associated with the explosive materials to the restraint RFID device 110. The restraint RFID device 110 transmits the information associated with the explosive materials to the proximate communications device 120 as transmitted sensor parameters 150.

The proximate communications device 120 may provide to the law enforcement officer in possession of the proximate communications device 120 the information associated with the explosive materials. The law enforcement office may instantly escalate the detainment activities of the suspect by alerting authorities of a legitimate bomb threat and escalating interrogation of the suspect to gather details of the bomb threat. The proximate communications device 120 may provide the information associated with the explosive materials to a remote processing system 420. The remote processing system 420 may be a centralized law enforcement system that can process the information associated with the explosive materials to gather further intelligence. The remote processing system 420 may store the information associated with the explosive materials in transmitted sensor parameter database 430 so the information associated with the explosive materials can be used in analysis of later investigations.

In another example, a shipping container is fitted with a restraint device that includes the nanotechnology sensors and the restraint RFID device 110. The nanotechnology sensors capture particles indicating that the shipping container has been exposed to a dangerous substance. Before unloading the shipping container from the shipping vessel, a shipping employee scans the proximate communications device 120 across the restraint RFID device 110 coupled to the shipping container. The restraint RFID device 110 transmits information associated with the dangerous substance to the proximate communications device 120 as transmitted sensor parameters 150.

The shipping employee is instantly alerted that the shipping container has been exposed to dangerous substances and initiates procedures to handle the shipping container. The shipping employee prevents the shipping container from being transferred to its next scheduled destination but rather detains the shipping container. Proximate communications device 120 communicates the information associated with the dangerous substances to the remote processing system 420. The remote processing system 420 may be a centralized port authority system that may analyze the information associated with the dangerous substances to gather further intelligence. The remote processing system 420 may store the information associated with the dangerous substances in the transmitted sensor parameter database 430 so that the information associated with the dangerous substances may be used in analysis of later investigations.

Embodiments of the invention may be implemented on but not limited to cervical collars, posey restraint systems, animal leashes/restraints, handcuffs for law enforcement, seat belts for adults, child restraint systems, seatbelts for airline passengers, and/or airport whole body scanners with wrist securing restraints.

Embodiments of the invention may be implemented on but not limited to trauma patients, patients in detox centers, animals recovering from surgery, dogs on a leash and out for a walk or having a so-called “shock collar” restraint device, passengers in a plane, Homeland Security Personnel arresting a suspect at the border, cargo shipments securely being readied for shipment across the world, a first time mother placing her infant in a car seat that would communication critical vital signs to the mother while she drives.

Embodiments of the invention may be compliant with the Patriot Act for homeland security maintaining privacy concerns. Embodiments of the invention may enable improved communication between governmental agencies regarding Homeland Security and terrorist targets. Embodiments of the invention may preemptively identify and engage Homeland Security prioritized analytes used by terrorists thereby preventing acts of terror before they occur. Embodiments of the invention may enable real time data dissemination to enable governmental savings via lower time to target. Embodiments of the invention may enable real time asset allocation to domestic and international locations based upon confirmed analyte discovery translating into government savings. Embodiments of the invention may be combined with body restraint systems that may be compliant with the Affordable Care Act and the drive towards electronic medical records improving efficiencies of the nurses and cost savings to hospitals. Embodiments of the invention may be combined with a cervical collar or posey restraint system that may be compliant with Medicare priorities of patient safety and lower incidence of skin ulcers and cost savings. Embodiments of the invention may be attached to a seat belt restraint system connected to the starter of a vehicle via a nanosensor alcohol switch that may lower death rates associated with drunk driving. Embodiments of the invention may be used as a driving under the influence (DUI) to lower the incidence of future drunk driving and enable the freedom of the driver.

While the present invention has been illustrated by the description of one or more exemplary embodiments, and while the embodiments have been described in considerable detail, they are not intended to restrict or in any way limit the scope of the appended claims to such detail. The various features discussed herein may be used alone or in any combination. Additional advantages and modifications will readily appear to those skilled in the art. The invention in its broader aspects is therefore not limited to the specific details, representative apparatus and method and illustrative examples shown and described. Accordingly, departures may be made from such details without departing from the scope or spirit of the general inventive concept. What is claimed is:

1. A restraint device, comprising:
at least a first restraint portion configured to be affixed to a wearer and restrain movement of the wearer;
at least one sensor on said first restraint portion, said sensor configured to sense at least one parameter associated with the wearer; and
a device for communicating with said at least one sensor and adapted to transmit to a remote location information related to the at least one sensed parameter.

2. The restraint device of claim 1, wherein said first restraint portion limits motion of a first body portion with respect to a second body portion of the wearer.

3. The restraint device of claim 1, wherein said first restraint portion limits motion of the wearer with respect to a defined location.

4. The restraint device of claim 1, wherein said sensor is configured to sense a physiological parameter associated with the wearer.

5. The restraint device of claim 4, wherein said sensor is positioned for contact with the skin of the wearer, wherein
the sensor is positioned within substantially direct contact of the skin of the wearer, or
the sensor is positioned within a close proximity of the skin of the wearer to adequately sense the physiological parameter associated with the wearer.

6. The restraint device of claim 4, wherein said sensor is configured to sense a virulent bacterial status of the wearer.

7. The restraint device of claim 4, wherein said sensor is configured to sense at least one of a skin temperature, a skin pH, a heart rate, or a blood oxygen saturation of the wearer.

8. The restraint device of claim 1, wherein said sensor is configured to sense the presence of, or exposure of the wearer to, at least one of a biological substance, a chemical substance, or a radioactive substance.

9. The restraint device of claim 8, wherein the biological or chemical substance is associated with at least one of an explosive material, a chemical weapon, or a dangerous biological substance.

10. The restraint device of claim 1, wherein said sensor comprises nanotechnology components adapted to sense the at least one parameter.

11. The restraint device of claim 1, further comprising an indicator for indicating detection of the at least one parameter.

12. The restraint device of claim 11, wherein said indicator is configured to illuminate upon detection of the at least one parameter.

13. The restraint device of claim 1, further comprising:
a device for receiving signals from said at least one sensor and adapted to store information related to the at least one sensed parameter.

14. The restraint device of claim 1, wherein the device is a Radio Frequency Identification (RFID) device configured to transmit information related to the at least one sensed parameter via RFID communication.

15. The restraint device of claim 14, further comprising:
a rectenna configured to convert electromagnetic energy generated by the RFID communication of the RFID device to direct current (DC) power to power the at least one sensor, the RFID device, and/or the device for receiving signals.

16. The restraint device of claim 14, wherein the RFID device is configured to transmit information related to the at least one sensed parameter to a proximate communications device that is within proximity of the RFID device to receive the transmitted information related to the at least one sensed parameter via the RFID communication.

17. The restraint device of claim 16, wherein the RFID device is further configured to select authorized information from the information related to the at least one sensed parameter, wherein the authorized information is authorized to be transmitted to the proximate communications device.

18. The restraint device of claim 17, wherein the RFID device is further configured to encrypt the authorized information from the information related to the at least one sensed parameter before transmitting the authorized information to the proximate communications device.

19. The restraint device of claim 14, wherein the RFID device is capable of transmitting the at least one sensed parameter via Near Field Communication (NFC).

20. The restraint device of claim 14, wherein the restraint device is a bracelet.

21. The restraint device of claim 19, wherein the RFID device that is coupled to the bracelet is further configured to transmit a location and/or a physiological parameter associated with the wearer to the proximate communications device to notify a caregiver of the wearer of the location and/or health of the wearer.

22. The restraint device of claim 1, further comprising:
a device for delivering an electrical charge to the wearer.

23. The restraint device of claim 1, wherein at least a portion of the restraint device is configured to illuminate to facilitate visibility in low-light conditions.

24. A proximate communications device, comprising:
a Radio Frequency Identification (RFID) device configured to receive information related to at least one sensor parameter via RFID communication from a RFID device coupled to a restraint device configured to restrain movement of a wearer of the restraint device, wherein the information related to at least one sensor parameter is associated with the wearer of the restraint device; and
a user interface configured to provide the information related to at least one sensor parameter to an end user associated with the proximate communications device.

25. The proximate communications device of claim 24, wherein the RFID device is further configured to receive the information related to at least one sensor parameter via RFID communication when the RFID device is within proximity of the RFID device coupled to the restraint device.

26. The proximate communications device of claim 25, wherein the RFID device is further configured to decrypt the information related to at least one sensor parameter received from the RFID device coupled to the restraint device.

27. The proximate communications device of claim 25, wherein the RFID device is further configured to provide the information related to at least one sensor parameter to a remote processing system via a network for processing of the information related to at least one sensor parameter.

28. The proximate communications device of claim 24, wherein the RFID device is further configured to receive information related to at least one sensor parameter via Near Field Communication (NFC) from the RFID device coupled to the restraint device.

29. A method of sensing at least one parameter associated with a wearer, comprising:
restraining the movement of the wearer using a restraint device having at least one sensor;
sensing the at least one parameter with the sensor; and
transmitting to a remote location information related to the at least one sensed parameter.

30. The method of claim 29, wherein sensing the at least one parameter comprises sensing a physiological parameter associated with the wearer.

31. The method of claim 29, wherein sensing the at least one parameter comprises sensing the presence of, or exposure of the wearer to, at least one of a biological substance, a chemical substance, or a radioactive substance.

32. The method of claim 29, further comprising:
indicating detection of the at least one parameter.

33. The method of claim 29, further comprising:
storing information related to the at least one sensed parameter.

34. The method of claim 29, further comprising:
transmitting information related to the at least one sensed parameter via Radio Frequency Identification (RFID) communication.

35. The method of claim 34, further comprising:
transmitting information related to the at least one sensed parameter to a proximate communications device that is within proximity of an RFID device coupled to a restraint device to receive the transmitted information related to the at least on sensed parameter via the RFID communication.

36. The method of claim 35, further comprising:
selecting authorized information from the information related to the at least one sensed parameter.

37. The method of claim 36, further comprising:
transmitting the authorized information to the proximate communications device.

38. The method of claim 34, further comprising:
transmitting information related to the at least one sensed parameter via Near Field Communication (NFC).
